(19) United States
(12) Patent Application Publication
(10) Pub. No.: US 2023/0001091 A1
(43) Pub. Date: Jan. 5, 2023

(54) INJECTION DEVICE

(71) Applicant: Sanofi, Paris (FR)

(72) Inventor: Stephan Riedel, Frankfurt am Main (DE)

(21) Appl. No.: 17/782,243

(22) PCT Filed: Nov. 30, 2020
(86) PCT No.: PCT/EP2020/083903

§ 371 Date: Jun. 3, 2022

(30) Foreign Application Priority Data
Dec. 11, 2019 (EP) 19306628.9

(51) Int. Cl. A61M 5/19; A61M 5/24

(52) U.S. Cl. CPC A61M 5/19; A61M 5/2448; A61M 5/284

(57) ABSTRACT

The present disclosure relates to an injection device for expelling of a dose of a medicament. The injection device includes an elongated housing with a distal end and a proximal end opposite to the distal end. The elongated housing has a body and a protective cap. A medicament container has a barrel having a first cavity in a proximal direction by a first stopper slidably arranged inside the barrel. The protective cap is configured to abut on a distally facing abutment portion of the barrel. The body is configured to abut on a proximally facing abutment portion of the first stopper. The first stopper is movable in distal direction relative to the barrel from an initial position into a priming position by moving the protective cap along the proximal direction relative to the body from a preset position into an activation position.

### CROSS REFERENCE TO RELATED APPLICATIONS

The present application is the national stage entry of International Patent Application No. PCT/EP2020/083903, filed on Nov. 30, 2020, and claims priority to Application No. EP 19306628.9, filed on Dec. 11, 2019, the disclosures of which are incorporated herein by reference.

### TECHNICAL FIELD

The present disclosure relates to an injection device for expelling of a dose of a medicament. The disclosure particularly relates to injection devices, such as a handheld injectors configured to expel a single or multiple doses of a fixed or variable size. In a further aspect the disclosure relates to injection devices configured to prepare a liquid medicament prior to conduct an injection procedure. Preparation of the medicament may include mixing of a powdered or lyophilized medicament with a diluent or solvent.

### BACKGROUND

Drug delivery devices for setting and dispensing a single or multiple doses of a liquid medicament are well-known in the art. Generally, such devices have substantially a similar purpose as that of an ordinary syringe.

Drug delivery devices, such as pen-type injectors have to meet a number of user-specific requirements. For instance, a patien with a chronic disease, such as diabetes, may be physically infirm and may also have impaired vision. Suitable drug delivery devices especially intended for home use therefore need to be robust in construction and should be easy to use. Furthermore, manipulation and general handling of the device and its components should be intelligible and easily understandable. Such injection devices should provide settings for subsequent dispensing of a dose of a medicament of variable size. Moreover, a dose setting, as well as a dose dispensing procedure, should be easy to operate and intuitive to use.

Typically, a drug delivery device comprises a housing or a particular cartridge holder, adapted to receive a cartridge at least partially filled with the medicament to be dispensed. The device further comprises a drive mechanism, usually having a displaceable piston rod to operably engage with a bung or piston of the cartridge. By means of the drive mechanism and its piston rod, the bung or piston of the cartridge is displaceable in a distal or dispensing direction and may therefore expel a predefined amount of the medicament via a piercing assembly, e.g., in the form of an injection needle, which is to be releasably coupled with a distal end section of the housing of the drug delivery device.

The medicament to be dispensed by the drug delivery device may be provided and contained in a multi-dose cartridge. Such a cartridge typically comprises a vitreous barrel sealed in distal direction by means of a pierceable seal and being further sealed in proximal direction by the bung. With a reusable drug delivery device, an empty cartridge is replaceable by a new one. In contrast to that, a disposable drug delivery devices may be entirely discarded when the medicament in the cartridge has been dispensed or used-up.

With some drug delivery devices, such as pen-type injection devices, a user has to set a dose of equal or variable size by rotating a dose dial in a clockwise or dose-incrementing direction relative to a body or housing of the injection device. For injecting and expelling a dose of a liquid medicament, the user has to depress a trigger or dose button in a distal direction and hence towards the body or housing of the injection device. Typically, the user uses their thumb for exerting a distally directed pressure onto the dose button, which may be located at a proximal end of the dose dial and the dose dial sleeve, while holding the housing of the injection device with the remaining fingers of the same hand.

With injection devices, such as handheld injectors provided with a prefilled multi-chamber or dual chamber medicament container, such as a dual-chamber cartridge, the medicament contained inside the cartridge may be prepared before an injection takes place. With a dual-chamber cartridge or dual-chamber syringe, for example, it may be necessary to conduct a priming step or a priming procedure, e.g., to mix the medicament with one of a solvent and a diluent in order to prepare the liquid medicament that is intended to be expelled and injected into biological tissue.

With other examples, a priming step may be necessary in order to assure that a plunger or piston rod is in abutment with a piston of the medicament container or cartridge. With some devices, it may be further necessary to conduct a so-called priming shot or air shot in order to expel eventual remaining air from the cavity of the medicament container.

With manually operable injection devices, it may be quite cumbersome and sometimes rather sophisticated for the end user to conduct such a priming step or priming procedure.

### SUMMARY

It is therefore desirable to provide an improved injection device for expelling of a dose of a medicament, wherein the injection device provides a rather user-friendly, efficient and failure safe approach to conduct a priming step or priming procedure. When the injection device is equipped with a dual-chamber cartridge that requires mixing or preparation of the liquid medicament the improved injection device should provide a rather easy, intuitive and failure safe approach to correctly conduct such a priming step or priming procedure.

In one aspect there is provided an injection device for expelling of a dose of a medicament. The injection device comprises an elongated housing. The elongated housing comprises a distal end and a proximal end. The proximal end is located opposite to the distal end. The elongated housing, typically extending in a longitudinal direction with a long axis, comprises a body and a protective cap. Typically, the protective cap is detachably connected to the body. The injection device further comprises a medicament container. The medicament container comprises a barrel having a first cavity. The first cavity is sealed in proximal direction or towards the proximal direction by a first stopper. The first stopper is slidably arranged inside the barrel. Typically, the medicament container is located inside the housing. The medicament container is typically located inside the body of the housing.

The protective cap is configured to abut on a distally facing abutment portion of the barrel. The body of the housing is configured to abut on a proximally facing abutment portion of the first stopper. The first stopper is movable in distal direction relative to the barrel from an initial position into a priming position. In other words, during a priming step or priming procedure the stopper is subject to a distally directed movement relative to the barrel from the initial or proximal position into the priming or distal position. The movement of the first stopper is induced or caused by the protective cap moving along the proximal direction relative to the body from a preset position into an activation position.

The protective cap, either typically located at the distal end of the housing and/or forming the distal end of the housing and the body, or typically located at the proximal end of the housing or forming the proximal end of the housing, may be moved closer together when moving from the preset position into the activation position. Hence, upon or during movement of the protective cap from the preset position towards and into the activation position, the total elongation of the elongated housing may be reduced. Since the protective cap is configured to abut on the distally facing abutment portion of the barrel the protective cap is operable to apply a proximally directed pressure onto the barrel. Likewise, the body is configured to abut on a proximally facing abutment portion of the first stopper. Hence, the body is operable to apply a distally directed pressure onto the first stopper.

In an initial configuration the protective cap may be in direct or indirect abutment with the distally facing abutment portion of the barrel. Likewise, the body may be direct or indirect abutment with a proximally facing abutment portion of the first stopper. Now and as the protective cap is moved relative to the body in proximal direction from the preset position into and/or towards the activation position the protective cap applies a proximally directed pressure onto the barrel while the body applies a distally directed pressure onto the stopper. As a consequence and when the respective forces are above a break loose force of the first stopper relative to the barrel the stopper becomes subject to a distally directed movement relative to the barrel.

When the protective cap is in the preset position relative to the body it does not necessarily be in direct or indirect engagement with the barrel. In the preset position also the body does not necessarily have to be in direct or indirect abutment with the first stopper. It is sufficient, when a longitudinal abutment between the protective cap and the distally facing abutment portion of the barrel is established during the movement of the body from the preset position into or towards the activation position. The same is valid for a direct or indirect abutment of the body with the first stopper.

In the preset position there may remain a functional gap between the body and the first stopper which is closed as the protective cap is moved relative to the body from the preset position towards the activation position.

In the present context an indirect abutment of the protective cap and the barrel means, that there may be provided one or several mechanical components between the protective cap and the barrel, wherein such components are configured to transfer a longitudinal force effect from the protective cap towards the barrel. The same applies for the indirect abutment between the first stopper and the body. Also here, there may be provided one or several mechanical components arranged between the body and the first stopper, which components are operable to transfer a mechanical pressure, force or thrust from the body towards and onto the first stopper.

In effect and by moving the protective cap from the preset position into the activation position relative to the body a priming procedure may be conducted by the end user rather easily and intuitively. During and due to the relative movement of the protective cap and the body the first stopper moves a well-defined distance relative to the barrel of the medicament container.

The priming distance, i.e., the distance the first stopper has to be moved from the initial position to reach the priming position directly correlates with the distance between the preset position and the activation position of protective cap and body. Typically, the priming distance is less than or equal to the activation distance, i.e., the distance the protective cap moves relative to the body from the preset position to the activation position.

With some examples, the activation position may be visually and/or haptically indicated on the outside of the injection device. Reaching of the activation position is therefore immediately apparent to the user from outside the housing of the injection device. In this way, an increased degree of patient safety and intuitive use of the injection device may be provided and supported.

The patient or end user receives a well-defined and intuitive feedback that the activation position of the protective cap, which is correlated with the priming position of the first piston has been reached and that priming has been conducted, or that a mixing of two components of a dual-chamber cartridge or syringe may be conducted.

According to a further example the injection device comprises an activation lock detachably or movably arranged on the elongated housing. The activation lock is operable to block or to prevent a movement of the protective cap from the preset position towards the activation position. The activation lock serves to prevent inadvertent activation or inadvertent priming of the injection device. A user of the injection device may first remove, detach or move the activation lock into a release configuration so as to enable a movement of the protective cap from the preset position towards or into the activation position.

With some examples the activation lock is movable relative to at least one of the protective cap and the elongated housing. The activation lock may be movably connected to at least one of the elongated housing and the protective cap. With some examples the activation lock is movable relative to both, the activation lock and the elongated housing.

With some examples the activation lock is pivotally or slidably supported on at least one of the protective cap and the elongated housing. It may be pivotable from a blocking position into a release position or release configuration. Here, a pivot axis may extend parallel to the elongation of the housing and/or along a central axis of the barrel.

When and as long as the activation lock is in the blocking position it effectively blocks and/or prevents a movement of the protective cap from the preset position towards the activation position, hence a movement of the protective cap in proximal direction and/or relative to the elongated housing.

With some examples the activation lock may be movably or detachably supported on one of a proximally facing abutment portion of the protective cap and a distally facing abutment portion of the elongated housing, typically of a body of the elongated housing.

According to another example the activation lock is frangibly connected to at least one of the body and the protective cap. A frangible activation lock may be provided as a tamper evident component, which is detachably and non-re-attachably connected to at least one of the body and the protective cap.

The frangible and non-re-attachable connection of the activation lock to at least one of the body and the protective cap may be beneficial in that a manipulated injection device with the frangible activation lock disconnected or detached may not be re-attached to the body or to the protective cap. In this way a detached or at least partially detached or removed activation lock is a direct indicator to the user, that the injection device has been manipulated. In this case, the injection device should not be used.

According to another example the activation lock is connected to at least one of the body and the protective cap by at least one of a perforated structure and a structurally weakened connector. The perforated structure and/or the structurally weakened connector comprises a predetermined breaking structure. In this way, the activation lock may be detached from the housing or it may be at least moved relative to the housing, e.g., by disrupting or by destroying the predetermined breaking structure.

With some examples the activation lock is integrally formed with at least one of the body and the protective cap. The protective cap and/or the body may comprise an injection molded plastic component.

With some examples the activation lock is provided with a detachment flap forming a gripping structure for the user in order to grip the activation lock and to disrupt or to disconnect the activation lock from the housing. With other examples, the flap may be used to move the activation lock into a release configuration, in which the protective cap is movable from the preset position into the activation position.

According to another example the activation lock comprises a ring. The ring encloses an outside surface of the housing. Hence, the ring may clasp around the housing. It may clasp at least one of the protective cap and the body. The ring may be located in an interface section of protective cap and body.

With some examples and when the activation lock is connected to both, the body and the protective cap and is further frangibly connected to at least one of the body and the protective cap may be secured or attached to the body by way of the activation lock. Destroying or disrupting the activation lock may then not only give way to move the protective cap from the preset position into the activation position but may also enable detachment of the protective cap from the body.

According to another example the activation lock comprises a distally facing abutment portion. The distally facing abutment portion is configured or operable to abut with a proximally facing abutment portion of the protective cap. When the distally facing abutment portion of the activation lock abuts with the proximally facing abutment portion of the protective cap the activation lock is effective to prevent or to block a proximally directed movement of the protective cap relative to the body.

In another example the activation lock comprises a proximally facing abutment portion. The proximally facing abutment portion is configured or operable to abut with a distally facing abutment portion of the body. In this way and when in a longitudinal abutment with the body the activation lock prevents and blocks a distally directed movement of the body relative to the protective cap.

With such examples, wherein the activation lock is provided as a separate piece detachably arranged on the housing it comprises a distally facing abutment portion and a proximally facing abutment portion. Here, the activation lock may act as a spacer between the proximally facing abutment portion of the protective cap and the distally facing abutment portion of the body. As long as the protective cap is located between the proximally facing abutment portion of the protective cap and the distally facing abutment portion of the body a respective movement from the preset position into the activation position is effectively blocked.

In the preset position the activation lock may be in abutment with at least one of the proximally facing abutment portion of the protective cap and the distally facing abutment portion of the body. However, it may be also arranged with a certain longitudinal gap to the abutment portions of the protective cap and the body, respectively.

With some examples the activation lock comprises numerous segments, e.g., numerous ring segments frangibly connected to each other, e.g., by way of a perforated structure or by a structurally weakened connector. Hence, in addition to or instead of detaching the activation lock from the housing the numerous segments of the activation lock, e.g., the ring segments, may be disconnected from each other, thereby destroying the integrity of the activation lock, e.g., thereby destroying the annular structure of the ring. Individual segments of the activation lock, e.g., individual ring segment thereof may remain connected to one of the protective cap and the body but may be moved, bended or pivoted in such a way, that they are outside a blocking engagement with one of the proximally facing abutment portion of the protective cap and the distally facing abutment portion of the body.

According to another example a proximally facing abutment portion of the protective cap or the above-mentioned proximally facing abutment portion of the protective cap is configured or operable to abut on a distally facing abutment portion of the body when the protective cap is in or reaches the activation position relative to the body. Also here, the distally facing abutment portion of the body may be the above-mentioned distally facing abutment portion of the body that is configured to abut with a proximally facing abutment portion of the activation lock.

In other words the proximally and distally facing abutment portions of the protective cap and the body intended and configured to engage or to abut with the proximally and distally facing abutment portions of the activation lock may also directly abut, when the protective cap and the body reach the activation position.

In this way the proximally and distally facing abutment portion of the protective cap and the body provide a twofold function. With the activation lock arranged between the proximally facing abutment portion of the protective cap and the distally facing abutment portion of the body the respective abutment portions of the protective cap and the body serve to prevent a movement of the protective cap from the preset position into the activation position. With the activation lock removed or with the activation lock in a release configuration the proximally facing abutment portion of the protective cap engaging or in abutment with the distally facing abutment portion of the body serves to delimit the movement of the protective cap from the preset position into the activation position.

Hence, the mutual abutment of the proximally facing abutment portion of the protective cap and the distally facing abutment portion of the body defines the activation position. In this way, the activation distance the protective cap moves relative to the body as well as the priming distance the first stopper moves relative to the barrel may be delimited. Insofar, a movement of the first piston from the initial position towards and beyond the priming position may be effectively prevented.

According to another example the protective cap and the body are telescopically displaceable in a longitudinal direction. The longitudinal direction typically extends along a long axis of the elongated housing. One of the protective cap and the body comprises a receptacle. The other one of the protective cap and the body comprises an insert portion. The receptacle is sized to receive the insert portion. On the other hand, the insert portion is sized to become inserted into the receptacle.

By providing a telescopic interface of protective cap and body, the protective cap may slide in longitudinal direction relative to the body. Moreover, the mutual arrangement of receptacle and insert portion provides a sliding engagement in longitudinal direction. The sliding engagement may also comprise a rotational guiding. Hence, the protective cap and the body may be subject to a longitudinal sliding motion and/or to a helical sliding motion.

Typically, the insert portion may be reduced in diameter compared to the receptacle. An outside surface of the insert portion has an outside diameter that is less or equal than an inside diameter of the sidewall of the receptacle. The diameter reduced or stepped down insert portion may further provide a mount for the activation lock.

According to a further example the activation lock is arranged on the insert portion. The activation lock, e.g., implemented as a ring may clasp around the insert portion. The activation lock may longitudinally abut with an end face of the sidewall of the receptacle. The activation lock may further longitudinal engage with a radially inwardly extending stop of the insert portion. The end face of the receptacle may provide one of the proximally facing abutment portion of the protective cap and the distally facing abutment portion of the body. The stepped down portion of the insert portion may provide the other one of the proximally facing abutment portion of the protective cap and the distally facing abutment portion of the body.

The activation lock may flush with an outside surface of a sidewall of the protective cap and/or of the body. When implemented as a ring the thickness of the ring may correspond to the radial step size of the insert. In this way, an outside surface of the ring may flush with the outside surface of that one of the body and the protective cap that is provided with the insert portion.

According to another example the protective cap is detachably fixed to the body via a fastening mechanism. The fastening mechanism is transferable from a locked configuration into a release configuration. When in the locked configuration the protective cap is fastened and/or fixed to the body. When in the release configuration the protective cap may be detached from the body.

With some examples the protective cap is detachably connected to the housing of the injection device, e.g., to the body and/or a sleeve surrounding or enclosing the medicament container. The fastening mechanism may provide a fastening feature on the protective cap correspondingly or complementary shaped counter fastening feature provided on the sleeve or body. With some examples, one of the fastening feature and the counter fastening feature comprises a radial protrusion and the other one of the fastening feature and the counter fastening feature comprises a radial recess or groove shaped and configured to engage with the protrusion.

The fastening feature and the counter fastening feature may provide a snap fit connection between the protective cap and the body or sleeve. Generally, the protective cap may be detached and disconnected from the body by overriding the fastening mechanism.

The fastening mechanism is and/or remains in the locked configuration when the protective cap is in the preset position. The fastening mechanism is in or reaches the release configuration when the protective cap is in the activation position or when the protective cap approaches or reaches the activation position.

By way of the fastening mechanism, a detachment of the protective cap may require a movement of the protective cap from the preset position to the activation position. Only when the protective cap has reached the activation position it may be removed from the body. In this way a user of the injection device is obliged to move the protective cap from the preset position into the activation position before the protective cap may be detached from the injection device in order to expel a dose of the medicament.

According to another example the fastening mechanism comprises a slot in one of a sidewall of the protective cap and the body. The fastening mechanism further comprises a pin protruding from the sidewall of the other one of the protective cap and the body. The pin is slidably displaceable along the slot. Typically, the pin is slidably engaged with the slot. The pin and the slot form a slotted link between the protective cap and the body. The fastening mechanism provides a forced guidance for the protective cap on the body.

According to a further example the slot comprises a dead end portion forming a stop for the pin.

The slot further comprises an escape portion to enable removal of the pin from the slot. The dead end portion extends at a predefined direction relative to the escape portion. Typically, the dead end portion and the escape portion merge into each other at a predefined angle. At least one or both of the dead end portion and the escape portion may comprise a rather straight shape. With regards to the longitudinal axis of the elongated housing, at least what of the escape portion, the dead end portion or an intermediate portion comprises a bending or curved structure.

The dead end portion comprises an end stop for the pin. The dead end stop of the dead end portion serves to delimit a movement of the pin beyond this dead end stop. When the slot is located in the body the dead end stop is located at a distal end of the dead end portion of the slot. When the slot is located or arranged in or on the protective cap the dead end stop is located or forms a proximal end of the dead end portion.

The dead end stop is that longitudinal end of the dead end portion that faces away from the escape portion. Hence, an opposite end of the dead end portion merges into the escape portion, e.g., via the intermediate portion. The escape portion comprises a free end portion. The free end portion of the escape portion is located at an end of the escape portion being furthest away from the dead end portion. That end of the escape portion merging with the dead end portion is located opposite to the free end portion of the escape portion. Once the pin is guided through the escape portion it may leave the slot through the free end portion.

In an initial configuration, hence when the protective cap is in the initial position the pin is typically located inside the dead end portion. It may be located near or even in abutment with the dead end stop. A movement of the protective cap in distal direction relative to the body is blocked by the pin engaging with the dead end stop.

During movement of the protective cap from the preset position into the activation position the pin is moved along the dead end portion. When arriving in the activation position the pin is in the intermediate portion or in the merging section between the dead end portion and the escape portion. So when the protective cap is in the activation position the pin has left the dead end portion.

A movement of the protective cap in distal direction relative to the body from the activation position leads to a respective guiding of the pin along the escape portion. The pin is then allowed to leave the slot and the fixing or fastening provided by the fastening mechanism is released or abrogated. Consequently, the protective cap may be detached and removed from the body.

With the above examples or with further examples the protective cap comprises a cup-shaped receptacle. The protective cap may comprise a tubular sleeve or tubular sheath sized and configured to receive or to accommodate a distal end of the injection device. With some examples, the protective cap comprises a closed distal end face. The protective cap comprises or forms a receptacle that is open in proximal direction so as to receive a distal end of the injection device or to receive a distal end of the housing of the injection device. Moreover, the proximal end, e.g., the proximal end of the sidewall of the protective cap may be provided with a fastening feature to engage with a correspondingly shaped counter fastening feature of the elongated housing or body of the injection device.

According to another example the medicament container comprises a second stopper to separate a second cavity from the first cavity. With this example the medicament container may comprise a dual chamber cartridge or dual-chamber syringe. The first and second stoppers are both slidably arranged inside the barrel. Between the first and the second stopper there is provided the first cavity. Between the second stopper and a distal outlet of the medicament container there is provided the second cavity. Typically, a liquid substance, such as a solvent or diluent is located inside the first cavity. In the second cavity there is typically provided a lyophilized or powdered pharmaceutical substance. The barrel may further comprise a bypass, e.g., in form of a radially outwardly protruding portion. In the region of the bypass, the inner diameter of the barrel is increased compared to regions located longitudinally outside the bypass.

In an initial configuration, the first stopper and the second stopper are located proximally of the bypass. During a priming procedure and by applying a distally directed pressure onto the first piston both pistons will be subject to a distally directed movement, e.g., until the second piston reaches the bypass. In this configuration the second piston is no longer able to seal the first cavity. Consequently, the liquid substance provided in the first cavity is allowed to enter the second cavity via the bypass.

The location of the bypass, the arrangement of the barrel inside the body as well as the priming distance and the activation distance are chosen in such a manner, that a majority or effectively the entirety of the liquid substance originally provided in the first cavity is transferred into the second cavity as the first stopper moves from the initial position into the priming position and/or as the protective cap moves from the preset position into the activation position.

Insofar and when the protective cap reaches the activation position the substance located inside the dual-chamber cartridge or dual-chamber syringe may be mixed, e.g., by shaking the injection device.

According to a further example at least one of a lyophilized medicament, a liquid medicament, a diluent end and a solvent is arranged inside the medicament container. In case that a powdery substance and a liquid substance are arranged inside the medicament container, the powdered substance, e.g., the lyophilized medicament is typically arranged in the second cavity whereas the liquid substance, e.g., the liquid medicament, the diluent and/or the solvent is arranged in the first cavity.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament may include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein may be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

### BRIEF DESCRIPTION OF THE FIGURES

The following description is with reference to the following Figures:

FIG. 1 schematically shows an example of an injection device,

FIG. 2 is a cross-section through the injection device of FIG. 1 in an initial or preset configuration,

FIG. 2A schematically illustrates the frangible connection of the activation lock,

FIG. 3 is a cross-section through the device of FIG. 2 with the activation lock removed,

FIG. 4 is a cross-section through the device of FIG. 3 after moving the protective cap into the activation position,

FIG. 5 shows the device of FIG. 4 after removal of the protective cap,

FIG. 6 shows the injection device after expelling of the liquid medicament,

FIG. 7 shows another example of an injection device in an initial configuration,

FIG. 8 shows another example of the injection device in a longitudinal cross-section,

FIG. 9 shows the device of FIG. 8 with the protective cap removed,

FIG. 10 shows the device of FIG. 9 with a sleeve in a retracted position during or for injecting of the medicament,

FIG. 11 is indicative of one example of a fastening mechanism to fix the protective cap to the body,

FIG. 12 shows the fastening mechanism with the protective cap approaching the activation position,

FIG. 13 shows the fastening mechanism during detachment of the protective cap from the body,

FIG. 14 is another example of the fastening mechanism,

FIG. 15 shows a further example of the fastening mechanism with the protective cap in the preset position,

FIG. 16 shows the fastening mechanism of FIG. 15 with the protective cap in the activation position,

FIG. 17 shows the first day mechanism of FIG. 16 during removal or detachment of the protective cap from the body,

FIG. 18 is a schematic longitudinal section of an exemplary embodiment of a medicament delivery device prior to use,

FIG. 19 is a schematic longitudinal section of an exemplary embodiment of a medicament delivery device prior to use,

FIG. 20 is a schematic perspective view of an exemplary embodiment of a container carrier,

FIG. 21 is a schematic longitudinal section of an exemplary embodiment of a medicament delivery device prior to use,

FIG. 22 is a schematic longitudinal detail section of an exemplary embodiment of a medicament delivery device during use,

FIG. 23 is a schematic longitudinal detail section of an exemplary embodiment of a medicament delivery device during use,

FIG. 24 is a schematic view of an exemplary embodiment of a medicament delivery device during interruption of delivery,

FIG. 25 is a schematic longitudinal section of an exemplary embodiment of a medicament delivery device after use, and

FIG. 26 is a schematic longitudinal detail section of an exemplary embodiment of a medicament delivery device after use.

### DETAILED DESCRIPTION

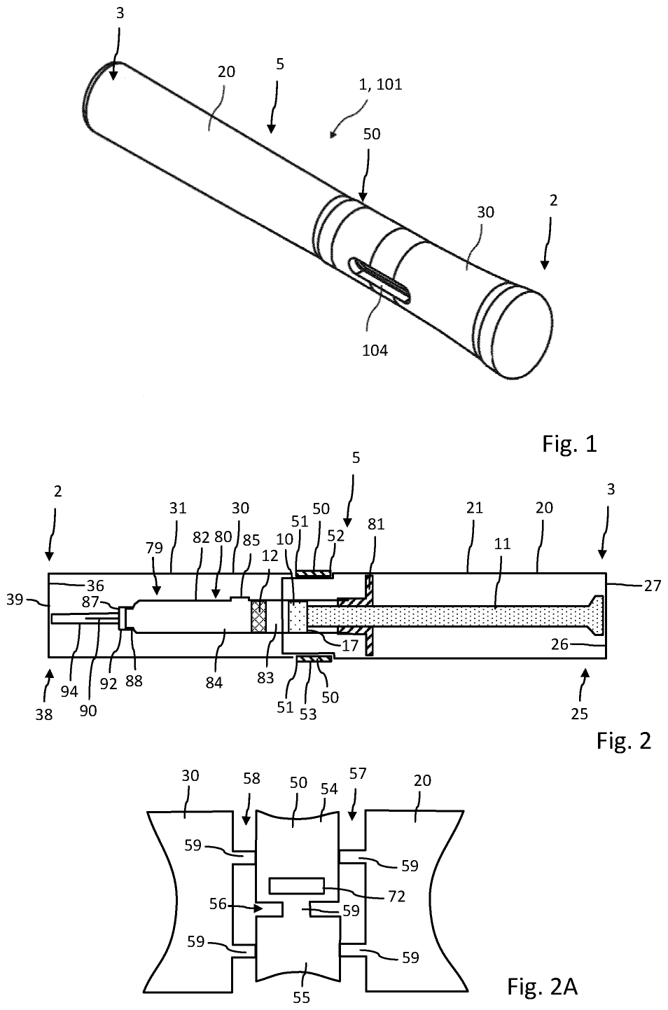

In FIG. 1, an example of a handheld injection device 1 is schematically illustrated. The injection device 1 may be configured as an injection pen. It may be also configured as a so-called auto injector. The injection device 1 comprises an elongated housing 5. The housing 5 may comprise a tubular shape. The housing 5 comprises a distal end 2 and an oppositely located proximal end 3. With its distal end 2 the injection device is configured to expel or to inject a liquid medicament located inside a medicament container 79 as illustrated in FIG. 2.

The medicament container 79 may be a component of a syringe 80. As illustrated in FIG. 2 the medicament container 79 comprises a barrel 82. The barrel is of tubular shape. The barrel 82 comprises an outlet 88 at a distal end. The outlet 88 may be provided with a needle hub 92. The needle hub 92 may be fixed to the outlet 88 of the barrel 82. The needle hub 92 is typically provided with an injection needle 90. The injection needle 90 may be covered by a needle shield 94. The needle shield 94 may comprise or provide an inner needle cap.

The barrel 82 further comprises a radially narrowing shoulder portion 86 towards the distal end. At least one of the shoulder portion 86, the outlet 88 and the needle hub 92 serves as or provides a distally facing abutment portion 87.

Inside the barrel 82 there are located a first stopper 10 and a second stopper 12. The sidewall of the barrel 82 and the two stoppers 10, 12 delimit or confine a first cavity 83. Inside the first cavity 83 there may be located a liquid substance, such as a solvent or diluent. There is provided a second cavity 84. The second cavity 84 may be provided with a lyophilized medicament or with a powdered substance. The second cavity 84 is delimited in proximal direction by the second stopper 12. It is delimited in distal direction by the outlet 88 of the barrel 82.

As further indicated in FIG. 2 the barrel 82 comprises a bypass 85. In the region of the bypass 85 The inner diameter of the barrel 82 is increased compared to longitudinal sections of the barrel 82 located offset from the bypass 85.

The syringe 80 further comprises a piston rod 11 or plunger. The piston rod 11 is configured to abut in longitudinal direction with an abutment portion 17 located or formed by a proximal end face of the first stopper 10. The barrel 82 is further connected to or mounted in a flange 81. By way of the flange 81 the barrel 82 is arranged and/or fixed inside the housing 5 of the injection device 1. As indicated in the FIGS. 1-10 the housing 5 comprises a proximal housing component, denoted as body 20. The housing 5 further comprises a distal housing component, denoted as protective cap 30. The protective cap 30 is detachably connected to the body 20. When assembled or attached to the body 20 the protective cap 30 covers the outlet end of the syringe 80.

In an initial and/or preset configuration as illustrated in FIG. 2 the distally facing abutment portion 87 of the barrel 82 is in direct or indirect longitudinal abutment with the protective cap 30. As illustrated in FIG. 2, the distal end 38 of the protective cap 30 comprises a closed end face 39 being in longitudinal or axial abutment with the needle shield 94. The needle shield 94 is further in direct abutment with the outlet 88 of the barrel 82. The end face 39 and hence the distal end 38 provide a proximally facing abutment 36 to engage with the distal end of the needle shield 94.

In a similar manner also the piston rod 11 is in direct abutment with a proximal end 25 of the body 20. Here, the body 20, which may be of tubular shape, comprises a closed end face 27 at a proximal end 25 in direct longitudinal abutment with a proximal end of the piston rod 11. Hence, the proximal end 25 and hence the proximal end face 27 of the body 20 forms a distally facing abutment 26 for the piston rod 11. The opposite distal end of the piston rod 11 is in abutment with the proximally facing abutment portion 17 of the first stopper 10. In this way, the body 20 is in indirect longitudinal abutment with the proximally facing abutment portion 17 of the first stopper 10. The protective cap 30 is in indirect longitudinal abutment with the distally facing abutment portion 87 of the barrel 82.

Instead of separate pieces that provide an effective abutment between the protective cap 30 and the barrel 82 or providing an abutment between the body 20 and the stopper 10 it is also conceivable, that the piston rod 11 is integrally formed with the body 20. Likewise, also the needle shield 94 may be integrally formed with or rigidly connected to the protective cap 30. Hence, the needle shield 94 may be alternatively provided by an inwardly extending protrusion of the distal end face 39 of the protective cap 30.

Similarly, also the piston 11 may be integrally formed with the body 20. It may be rigidly connected to the body 20.

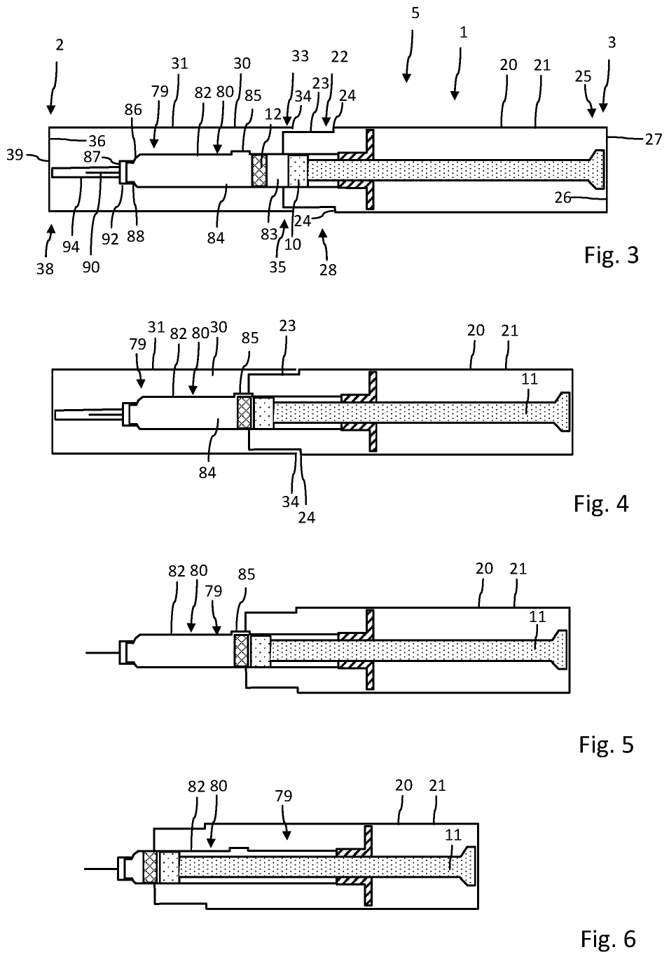

When in the initial configuration as illustrated in FIG. 2 there is provided an activation lock 50 between a proximal end 35 of the protective cap 30 and a stepped portion 22 of the body 20. As it becomes apparent from FIGS. 2-4 the protective cap 30 comprises a receptacle 33 at the proximal end 35. The receptacle 33 is open towards the proximal direction and is sized to receive an insert portion 23 of the body 20. The insert portion 23 of the body 20 is a stepped down portion. Hence, the sidewall 21 of the body 20 proximally adjacent to the insert portion may comprise an outer diameter or outer structure that is somewhat equivalent or even identical to the outside diameter or structure of the sidewall 31 of the protective cap 30. The stepped down portion 22 and hence the diameter-reduced insert portion 23 is sized to become slidably received inside the receptacle 33 as it is apparent from a comparison of FIGS. 3 and 4.

As long as the activation lock 50 is located in the interface of the telescopic engagement of the protective cap 30 and the body 20 the protective cap 30 is hindered to move in proximal direction relative to the body 20. As indicated in FIG. 2A the activation lock 50 is frangibly connected to at least one of the body 20 and the protective cap 30. The activation lock 50 may comprise an annular structure and may fill or traverse a longitudinal gap between the proximal end 35 of the protective cap 30 and the stepped down portion 22 of the body 20. As shown in FIG. 2 the activation lock 50 comprises a distally facing abutment portion 51, hence a distal end face to abut or to engage with a correspondingly shaped proximally facing abutment portion 34 of the protective cap 30.

The proximally facing abutment portion 34 may be provided by an end face of the sidewall 31 of the receptacle 33. In the opposite direction the activation lock 50 comprises a proximally facing abutment portion 52. The abutment portion 52 may be provided by a distal end face of the receptacle 33 forming the activation lock 50. The stepped down insert portion 23 is delimited in proximal direction by a distally facing abutment portion 24 of the body 20. As indicated in FIG. 2 the proximally facing abutment portion 52 of the activation lock 50 is in abutment with the distally facing abutment portion 24 of the body 20. In this way the activation lock fills or traverses the gap between the distally facing abutment portion 24 and the proximally facing abutment portion 34 of the body 20 and the protective cap 30, respectively.

As indicated in FIG. 2A the activation lock 50 comprises a ring 53 with numerous individual segments 54, 55, e.g., implemented as ring segment 54, 55. The segments 54, 55 are mutually connected by a perforated structure 56. In addition, at least one of the segments 54, 55 is connected to at least one sidewall 21, 31 of the body 20 or protective cap 30, respectively. Hence, the activation lock 50 may be detachably and frangibly connected to the protective cap 30 by a perforated structure 58. It may be further detachably or frangibly connected to the body 20 via the perforated structure 57. The perforated structure(s) 56, 57, 58 may each comprises one or more separate connectors 59.

Optionally, the activation lock 50 may be provided with a gripping flap 72 that facilitates an easy gripping and handling of the activation lock by a user. By gripping of the e.g., radially outwardly protruding gripping flap a user may easily invoke and induce a disruption of at least one of the perforated structures 56, 57, 58 thus enabling to detach or to remove the activation lock 50 of the housing 5.

The distal end 28 of the body 20 is provided with an insert portion 23.

The resulting configuration after detachment and removal of the activation lock 50 is illustrated in FIG. 3. Starting from the initial or preset position a user may now move the protective cap 30 relative to the body 20 in proximal direction. Due to the longitudinal or axial abutment of the protective cap 30 with the distally facing abutment portion 87 of the barrel 82 and due to the abutment of the proximally facing abutment portion 17 of the first stopper 10 with the body 20 the first stopper 10 will be subject to a distally directed movement relative to the barrel 82 as the protective cap 30 and the body 20 approach the activation position as illustrated in FIG. 4.

Since the first cavity 83 is filled with a substantially non-compressible medium a respective movement of the first stopper 10 relative to the barrel 82 equally transfers to a respective movement of the second stopper 12. As shown in FIG. 4, the second stopper 12 is moved into the region of the bypass 85. Consequently, the liquid substance initially located inside the first cavity 83 is transferred into the second cavity 84 via the bypass. In this configuration the medicament located inside the medicament container 79 can be mixed and/or prepared for a subsequent injection procedure.

The proximally directed movement of the protective cap 30 relative to the body 20 is delimited by the proximally facing abutment portion 34 of the protective cap 30 getting in abutment with the corresponding shaped distally facing abutment portion 24 of the body 20. In this way, the movement from the preset position into the activation position is stopped.

FIG. 5 is illustrative of the configuration of the injection device 1 after the protective cap 30 has been removed. The device 1 then ready for dispensing of the liquid medicament located inside the second cavity 84. FIG. 6 is indicative of a final configuration of the injection device 1 after dispensing or expelling of the medicament from the second cavity 84. During and/or for expelling of the medicament, the flange 81 and hence the barrel 82 is subject to a proximally directed movement relative to the body 20. In other words, the body 20 and/or the piston rod 11 is subject to a distally directed movement relative to the barrel 82, thereby moving both, the first and the second stoppers 10, 12 in distal direction. The liquid substance previously contained in the second cavity 84 is expelled via the injection needle 90.

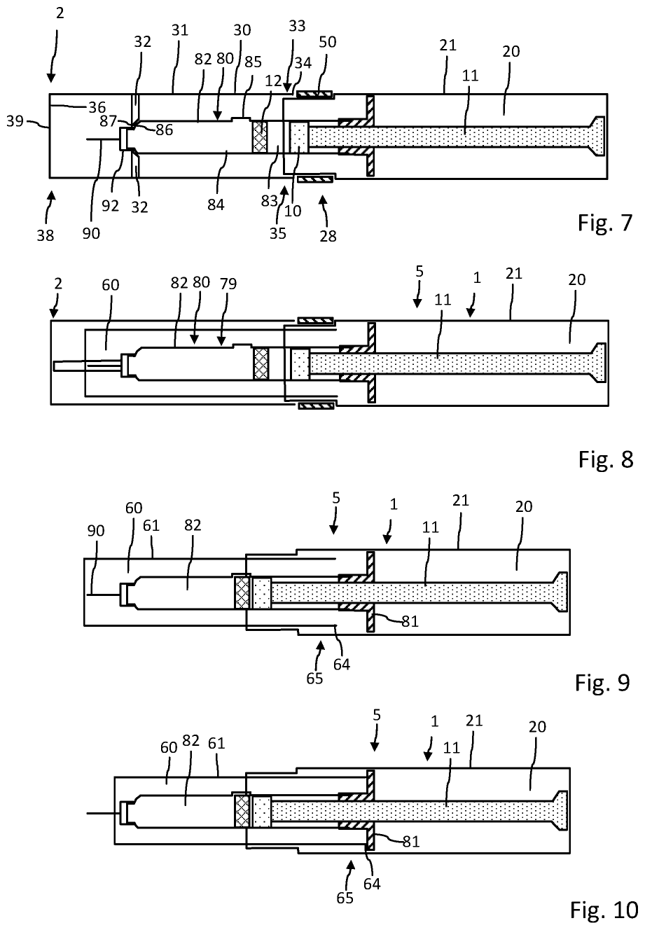

In FIG. 7 another example of an injection device 1 is shown. This injection device 1 is highly similar to the injection device as described and explained with regard to FIGS. 2-6. There is only one difference, namely that the protective cap 30 comprises an abutment portion 32 protruding radially inwardly from the sidewall 31 of the protective cap 30. The abutment portion 32 may comprise a radially inwardly extending flange configured to engage with the shoulder portion 86 of the barrel 82. Here, the shoulder portion 86 effectively provides the distally facing abutment portion 87 of the barrel 82.

In FIGS. 8-10 another example of an injection device 1 is illustrated. Again, this example is somewhat similar to the example as illustrated in FIGS. 2-6. But here, the body 20 further comprises a sleeve 60 displaceably, e.g., slidably arranged to the proximal portion of the body 20. The sleeve 60 is a kind of a needle sheath a needle shield. When the protective cap 30 is removed as illustrated in FIG. 9, the sleeve 60 form a distal end of the body 20. The distal end 68 of the sleeve 60 is located distally from the tip of the injection needle 90. In this way, a stitch danger may be effectively reduced.

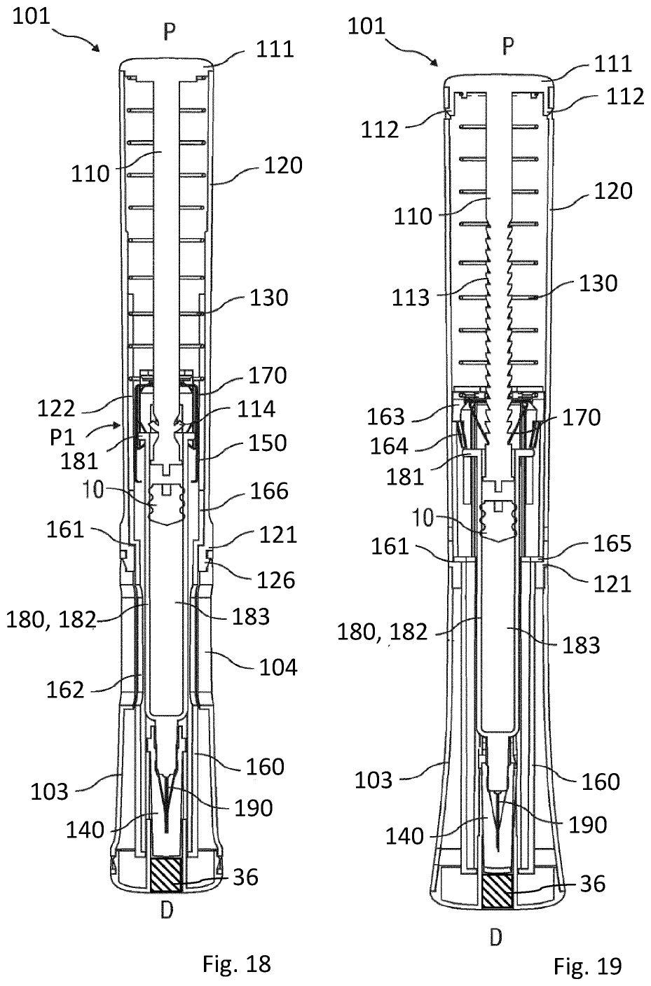

For conducting an injection, the distal end 68 of the sleeve 60 is pressed against the skin of the patient and the proximal portion of the body 20 is moved in distal direction relative to the sleeve 60 thus inducing a respective forward movement of the syringe 80 or barrel 82. This is schematically illustrated in FIG. 10. A rather detailed example of such an injection mechanism is further explained below with regard to FIGS. 18-26.

As becomes further apparent from FIGS. 9 and 10 a proximal end 65 of the sleeve 60 comprises an abutment 64 configured to engage with the flange 81 or with the barrel 82 of the syringe 80. In this way a distally directed movement of the barrel 82 and hence of the syringe 80 with the injection needle 90 mounted thereon in distal direction relative to the sleeve 60 can be delimited, namely when the abutment 64 at the proximal end 65 of the sleeve 60 abuts against a distally facing abutment portion of the flange 81. Here, and in the course of expelling of the medicament the sleeve 60 provides an effective abutment for the flange 81 relative to the skin of the patient. A holding force for the flange relative to the distally moving piston rod 11 can be provided by the abutment of the sleeve 60 on the patient's skin. The sleeve 60 is a component of the body 20. Insofar, the above-described abutment of the protective cap 30 may equally apply to the sleeve 60.

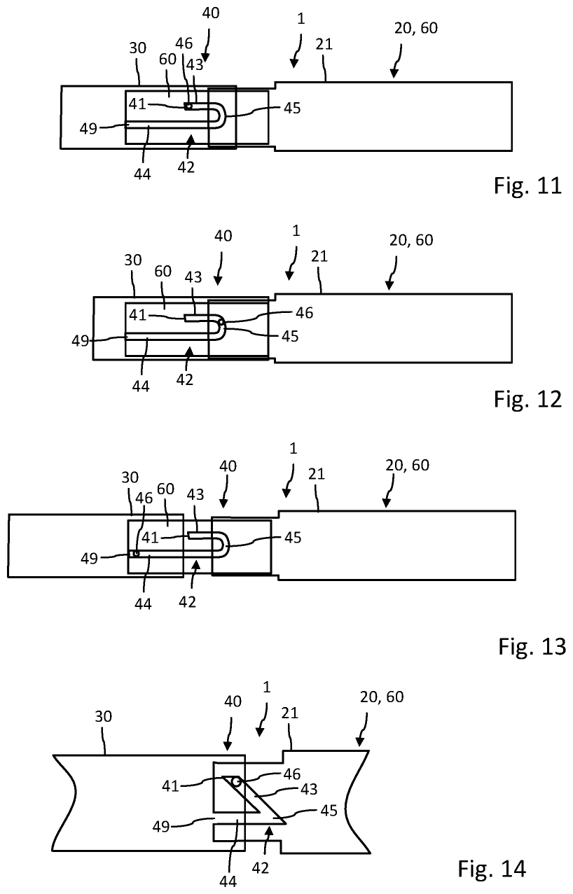

In FIGS. 11-13 one example of a fastening mechanism 40 is illustrated. The fastening mechanism 40 is active between the protective cap 30 and the body 20. The fastening mechanism 40 may be implemented as a slotted link. In the example as illustrated, the inside facing portion of the sidewall 31 of the protective cap 30 comprises a radially inwardly protruding pin 46 engaged with a slot 42 provided on an outside surface of the sidewall 21 of the body 20. In the example of FIGS. 11-13, the slot 42 is provided on an outside facing portion of a sidewall 61 of the sleeve 60. The sleeve is hindered to separate distally from the proximal portion of the body 20. From the configuration as illustrated in FIG. 9 it may only move in proximal direction.

The slot 42 comprises a dead end portion 43, an intermediate curved portion 45 and an escape portion 44. The escape portion 44 is open towards the distal direction. It comprises a free end portion 49 and enables a removal of the pin 46 as it moves from the intermediate portion 45 through the escape portion 44 as illustrated in FIG. 13. The dead end portion 43 comprises a dead end stop 41 located in a longitudinal end of the slot 42 opposite to the free end portion 49.

In the initial configuration as illustrated in FIG. 11 the pin 46 is located in the dead end portion 43. Upon and during movement of the protective cap 30 towards and into the activation position, which is illustrated in FIG. 12 the pin 46 is located in the intermediate portion 45. The pin 46 may enter the escape portion 44 only via the intermediate portion 45. Insofar, a detachment of the protective cap 30 from the body 20 is only possible by initially moving the protective cap 30 from the preset position as shown in FIG. 11 into the activation position. Only from the activation position, in which e.g., the medicament is appropriately mixed inside the syringe 80 the protective cap 30 can be detached by transferring the pin 46 from the intermediate portion 45 into the escape portion 44.

In FIG. 14 another example of a fastening mechanism 40 is illustrated. Also here, the fastening mechanism 40 comprises a dead end portion 43 with a dead end stop 41 and an escape portion 44. As illustrated in FIG. 14, the escape portion 44 extends in longitudinal direction. It runs substantially parallel to a longitudinal center axis of the housing 5. The dead end portion 43 is somewhat slanted or angled. In this way, the dead end portion 43 and the escape portion 44 mutually emerging in the intermediate portion 45 form a kind of a bayonet coupling for the pin 46. Hence, for the pin 46 sliding along the dead end portion 43, the protective cap 30 is subject to a helical movement, i.e., a combined rotational and longitudinal displacement relative to the body 20. Such a bayonet type coupling provided by the fastening mechanism 40 may be even beneficial to increase the longitudinal force exerted by the body 20 and the protective cap 30 to move the first stopper 10 and/or the second stopper 12 in distal direction relative to the barrel 82.

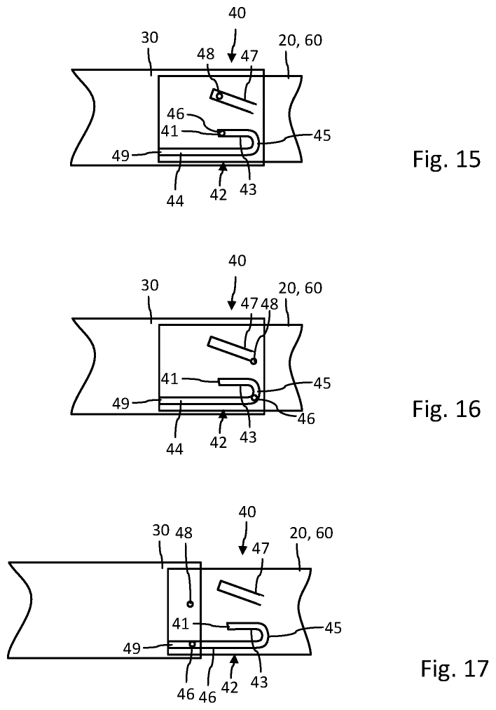

In the sequence of FIGS. 15-17 another example of a fastening mechanism 40 is described. Here, there are provided two separate slots 42, 47, each of which serving as a slotted link for a respective pin 48, 46. The slot 42 is somewhat equivalent to the slot 42 as illustrated in connection with FIGS. 11-13. The further slot 47 is somewhat skewed or slanted relative to the elongation of the dead end portion 43 and the escape portion 44 of the other slot 42. In this way and as the protective cap 30 is moved from the preset position as indicated in FIG. 15 towards and into the activation position as shown in FIG. 16 the slanted slot 47 induces an angular momentum or torque onto the protective cap 30 that leads to a respective tangential movement of the pin 46 through the intermediate portion 45 so as to ensure that the pin 46 is transferred from the dead end portion 43 into the escape portion 44 as the protective cap 30 reaches the activation position. The slanted slot may induce a flexible deformation of the protective cap as it moves towards the activation position. The tangential distance between the pins 46, 48 make radially reduce during this movement. As soon as the activation position has been reached, the mechanical biasing or tensioning may relax, thereby transferring the pin 46 into the escape portion 44.

In the example of FIGS. 18-26 an injection mechanism and the assembly of a syringe 180 inside a body 120 of a further injection device 101 is illustrated. With these illustrations the above-described mutual displacement of the protective cap 103 relative to the body 120 from the preset position into the activation position is not shown in detail. Moreover, the syringe 180 as illustrated in FIGS. 18-26 only comprises a single cavity 183.

The injection device 101 as illustrated in FIGS. 18-26 comprises a cap 103 that may include a viewing window 104 (e.g., an opening or a substantially transparent piece of material). The cap 103 may further include one or more indicia for providing a visual and/or tactile indication of use (e.g., which end of the device 101 should be applied to an injection site, etc.). For example, in an exemplary embodiment, the indicia include one or more arrows (painted or embossed) pointing toward a distal end of the device 101.

The injection device 101 is shown with the cap 103 already being in the activation position. As particularly illustrated in FIGS. 18 and 19, the cap 103 comprises longitudinally inwardly protruding abutment 36 engaged with the needle shield 140 thereby exerting a proximally directed pressure onto the barrel 182.

The activation lock 50 as illustrated and described in connection with FIGS. 1-17 is equally applicable to and/or usable with the injection device 101. The activation lock 50 may be arranged between a proximally facing abutment portion of the protective cap 103 and a distally facing abutment portion of the body 120. In effect, all features, examples and effects of the activation lock as described above are equally a correspondingly applicable to the injection device 101 as described in connection with FIGS. 18-26.

FIGS. 18 and 19 show a longitudinal section of an exemplary embodiment of such a medicament delivery device 101. A sleeve 160 is slidably coupled to the body 120. For example, the sleeve 160 may be telescoped within the body 120. In an exemplary embodiment, the sleeve 160 comprises one or more lateral sleeve windows 162 adapted to axially align with the viewing windows 104 (e.g., an opening or a substantially transparent piece of material) in the cap 103 when the sleeve 160 is in an extended position (as shown in FIG. 18) relative to the body 120.

In an exemplary embodiment, a container carrier 170 slidably disposed in the body 120. The container carrier 170 is adapted to retain a medicament container, e.g., a syringe 180, an ampoule, a cartridge, etc. For example, the syringe 180 includes a syringe barrel 182 arranged as a hollow cylinder defining a cavity 183 for receiving a medicament. A needle 190 is arranged at a distal end of the syringe barrel 182 in a manner to be in fluid communication with the cavity 183. A stopper 10 is disposed within the syringe barrel 182 for proximally limiting the cavity 183. The stopper 10 may be displaced within the syringe barrel 182 for ejecting the medicament from the cavity 183 through the needle 190.

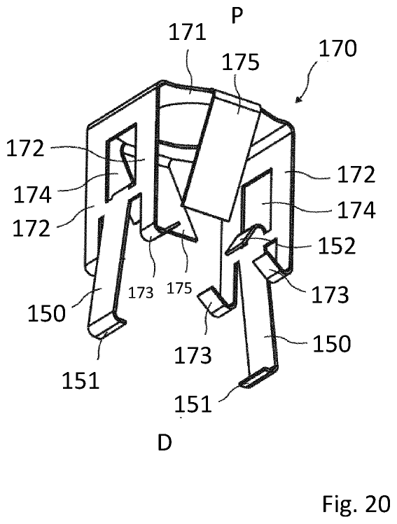

FIG. 20 shows an exemplary embodiment of a container carrier 170 according to the present disclosure. The container carrier 170 comprises a proximal portion 171 One or more first legs 172 extend distally from the proximal portion 171 In an exemplary embodiment, the first leg 172 comprises a radially inwardly directed first protrusion 173 arranged on a distal end of the first leg 172 and an aperture 174 (or recess). One or more second legs 175 extend distally from the proximal portion 171. The second leg 175 is biased radially inward.

In an exemplary embodiment, the container carrier 170 comprises one or more clamps 150 with a distal hook 151 and a proximal hook 152, wherein the proximal hook 152 is adapted to proximally engage a medicament container.

Referring again to FIGS. 18 and 19, the body 120 comprises one or more longitudinal first ribs 122 having a first axial length and adapted to radially outwardly abut the clamps 150 when the carrier 170 is in a first position P1 relative to the body 120. When the carrier 170 is moved from the first position P1 in the proximal direction P the clamps 150 slide along the first ribs 122.

In an exemplary embodiment, the sleeve 160 comprises one or more sleeve legs 166 extending in the proximal direction P beyond a collar 161. The sleeve legs 166 are adapted to radially outwardly support the clamps 150 such that they cannot deflect radially outwards depending on the axial position of the sleeve 160 relative to the clamps 150. The sleeve 160 comprises a proximal stop 164 and a distal stop 165. The proximal stop 164 is adapted to engage the sleeve 160 for limiting axial movement of the sleeve 160 in the distal direction D. In an exemplary embodiment the distal stop 165 may be part of the collar 161.

In an exemplary embodiment, a piston rod 110 is arranged within the body 120 in a manner to engage the stopper 10 for displacing it within the syringe barrel 182. In an exemplary embodiment, the piston rod 110 is attached to the body 120, preventing relative movement between the piston rod 110 and the body 120. In an exemplary embodiment the piston rod 110 may be integrally shaped with the body 120, or in another exemplary embodiment, the piston rod 110 may be secured to the body 120 by latches 112. In an exemplary embodiment, prior to use, an axial gap may be present between a distal end of the piston rod 110 and a proximal end of the stopper 10. The axial gap may prevent force being applied to the stopper 10 prior to use.

In an exemplary embodiment, a spring 130 is arranged over the piston rod 110 between a proximal end face 111 of the piston rod 110 and a spring seat 163 on the sleeve 160 thus biasing the sleeve 160 in the distal direction D relative to the piston rod 110 and body 120.

In an exemplary embodiment, the piston rod 110 comprises a ratchet toothing 113 adapted to be engaged by the second legs 175 of the carrier 170 in such a manner that the carrier 170 can move in the proximal direction P but is prevented from moving in the distal direction D relative to the piston rod 110.

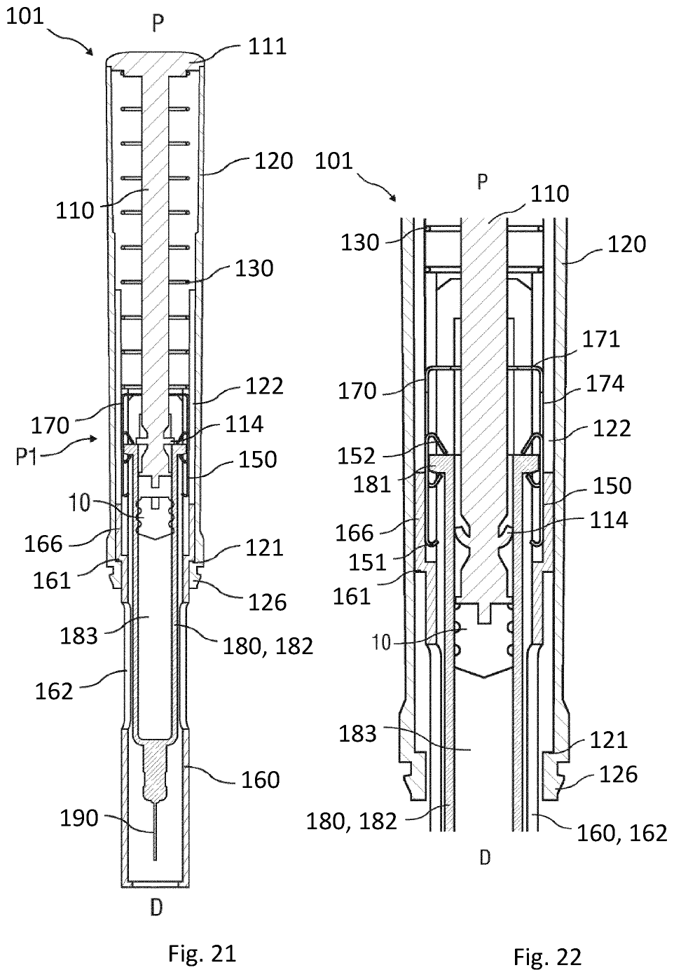

In an exemplary embodiment, a syringe retarder 114 is provided on the piston rod 110. The syringe retarder 114 comprises one or more resilient arms which in a relaxed state define a diameter greater than an internal diameter of the syringe barrel 182. In the initial state shown in FIG. 21, the syringe retarder 114 abuts the proximal end of the syringe barrel 182 such that the syringe 180 cannot move in the proximal direction P relative to the piston rod 110. On application of a predefined force to the syringe 180 in the proximal direction P relative to the piston rod 110, the resilient arms may be deflected radially inward, such that the piston rod 110 can enter the syringe barrel 182 thus allowing movement of the piston rod 110 relative to the syringe 180 in the distal direction D.

In an exemplary embodiment, a protective needle shield 140 is arranged over the needle 190. The cap 103 is adapted to engage the needle shield 140, e.g., by a barb, in manner to remove it from the needle as the cap 103 is removed from the body 102 by pulling it in the distal direction D. A snap feature 126 may be arranged on the body 102 for releasably snap fitting the cap 103 to the body 102.

In order to perform an injection, the medicament delivery device 101 may be operated according to the following exemplary method.

The cap 103 is pulled in the distal direction D relative to the body 102 thereby also pulling the protective needle shield 140 off the needle 190. The syringe 180 is prevented from moving in the distal direction D as its proximal flange 181 abuts the first protrusion 173 of the carrier 170 being in the first position P1. The carrier 170 is prevented from moving in the distal direction D by the second legs 175 being engaged to the ratchet 113. The collar 161 on the sleeve 160 distally abuts an axial stop 121 on the body 102 such that the sleeve 160 is also prevented from moving in the distal direction D.

FIG. 21 is a schematic longitudinal section of the medicament delivery device 101 after removal of the cap 103 and the protective needle shield 140. The needle 190 is located within the sleeve 160 preventing a user from touching and seeing it. In this state the medicament delivery device 101 may be held at the body 102 and the sleeve 160 may be pushed against an injection site, e.g., a patient's skin. Consequently, the sleeve 160 moves in the proximal direction P relative to the body 102 against the force of the spring 130, as the spring 130 is applying a biasing force on the spring seat 163 of the sleeve 160. Due to the movement of the sleeve 160 in the proximal direction P, the spring 130 is compressed. The syringe 180 remains in position relative to the body 102 due to the syringe retarder 114. As the syringe 180 and the needle 190 stay in position relative to the body 102 while the sleeve 160 moves in the proximal direction P, the needle 190 protrudes beyond a distal end of the sleeve 160 and is inserted into the injection site. Once the distal stop 165 abuts the flange 181 on the syringe 180, movement of the sleeve 160 relative to the syringe 180 and needle 190 stops. The needle 190 has reached its insertion depth. Any further movement of the sleeve 160 relative to the body 102 in the proximal direction P hence causes deformation of the syringe retarder 114, and the container carrier 170 moves proximally out of the first position P1 relative to the body 102.

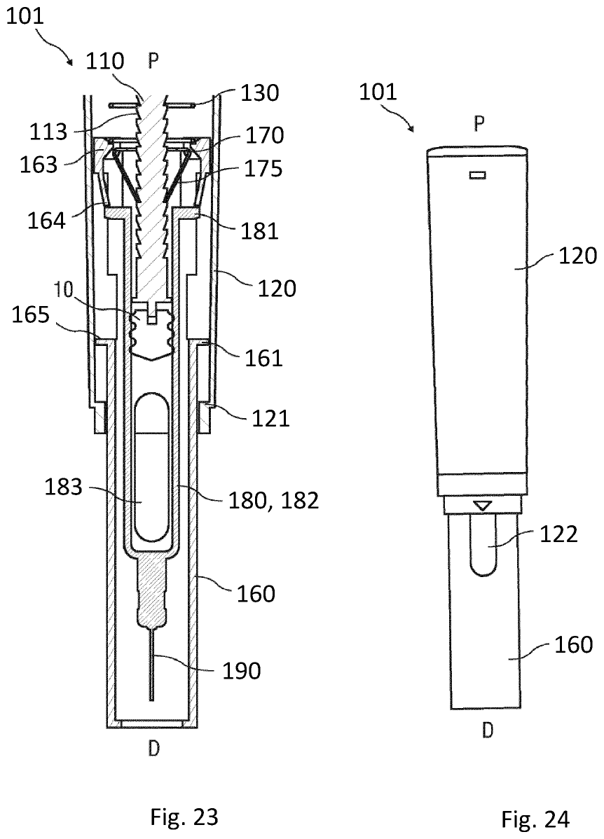

FIGS. 22 and 23 are schematic longitudinal detail sections in different section planes of the medicament delivery device 101 with the syringe 180 having moved in the proximal direction P relative to the piston rod 110. The increase in force required to deform the syringe retarder 114 may be a tactile feedback experienced by a user. As the piston rod 110 is coupled to the body 102, movement of the syringe 180 in the proximal direction P causes the piston rod 110 to abut the stopper 10 and displace it within the syringe barrel 182 ejecting the drug from the cavity 183 through the needle 190 into the injection site. As the sleeve 160 keeps moving relative to the body 102 the spring 130 is further compressed thus increasing its force according to Hooke's law thus providing a tactile feedback to the user about the state of the injection. As the syringe 180 moves in the proximal direction P relative to the body 102 and the piston rod 110, the flange 181 engages the proximal hook 152 within the carrier 170. Hence, the carrier 170 is also moved in the proximal direction P relative to the piston rod 110 thereby causing the second legs 175 to move along the ratchet toothing 113. As the second legs 175 disengage and engage successive teeth in the ratchet, an audible feedback may be provided in the form of a “click” sound.

If the medicament delivery device 101 was removed from the injection site after use, the spring 130 would return the sleeve 160 in the distal direction D, as in FIG. 23, until the proximal stop 164 of the sleeve 160 abuts the flange 181. As the second legs 175 of the carrier 170 are engaged in the ratchet toothing 113 the carrier 170, the syringe 180 and the needle 190 are prevented from returning in the distal direction D. The needle 190 would be hence covered again by the sleeve 160 despite the changed axial position of the syringe 180 relative to the body 102.

FIG. 24 is a schematic view of the medicament delivery device 101 having been removed from the injection site after partial delivery of the medicament. The overall length of the medicament delivery device 101 is shorter than in the initial state and the sleeve windows 162 are partially hidden within the body 102 thus providing a visual feedback as to the progress of the injection and as to the state of use of the device 101.

If the medicament delivery device 101 is in this state it can be re-applied against the injection site and the injection can be continued. The sleeve 160 will again move relative to the syringe 180 thus inserting the needle 190 into the injection site before the sleeve 160 abuts the syringe 180 moving it relative to the body 102 and piston rod 110 for delivering the drug thereby also moving the clamps 150 and the carrier 170.

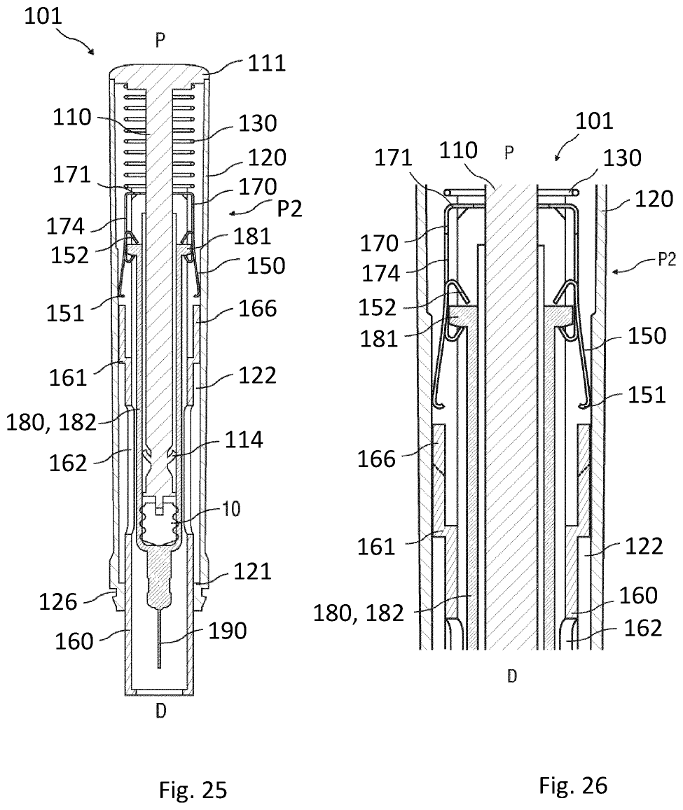

If after the initial start or after restart of the injection the body 102 is at least nearly fully depressed relative to the sleeve 160, the piston rod 110 will displace the stopper 10 until the stopper 10 bottoms out within the syringe 180 thereby at least nearly fully emptying the cavity 183. At this point the force opposing the movement of the body 102 in the distal direction D relative to the sleeve 160 considerably increases indicating to the user that the injection is finished. At this point the container carrier 170 has travelled to or beyond a second position P2 such that the clamps 150 could deflect radially outwards. However, as long as the sleeve 160 is maintained depressed the sleeve legs 166 still outwardly support the clamps 150 preventing their outward deflection. FIG. 25 is a schematic longitudinal section of the medicament delivery device 101 with the carrier 170 in the second position P2. FIG. 26 is a schematic longitudinal detail section of the medicament delivery device 101 with the carrier 170 in the second position P2, wherein the sleeve 160 is shown transparently to allow seeing the rib 122.

When the medicament delivery device 101 is been removed from the injection site, the spring 130 returns the sleeve 160 in the distal direction D relative to the carrier 170, syringe 180 and needle 190 until the proximal stop 164 on the sleeve 160 abuts the flange 181 of the syringe 180 thereby axially removing the sleeve legs 166 from the clamps 150 allowing them to radially outwardly deflect. Due to the movement of the sleeve 160 relative to the carrier 170, the needle 190 is covered again by the sleeve 160. On an attempt to again move the sleeve 160 in the proximal direction P relative to the body 102, the sleeve legs 166 would axially abut the outwardly deflected clamps 150 preventing re-exposure of the needle 190. The length of the first rib 122 can be modified in order to adapt the position and hence the percentage of drug delivered at which the clamps 150 are allowed to deflect radially outwards and lock out the sleeve 160.

The cap 103 of the medicament delivery device 101 serves for keeping the needle sterile prior to use, for removing the protective needle shield 140, for preventing unintended use of the medicament delivery device 101 prior to removal of the cap 103 and for providing rigid packaging.

The medicament delivery device 101 allows for application by a user, e.g., a patient or caregiver, wherein the body 102 can be held in one hand. The needle 190 of the medicament delivery device 101 is hidden from view during all states of operation.

The forces required to insert the needle 190 into the injection site and to deliver the drug can be adjusted by respectively selecting the spring 130, wherein the force for delivering the drug depends on the spring 130 and on the characteristics of the syringe 180, stopper 10, needle 190 and drug as well as on the friction between the syringe retarder 114 on the inner wall of the syringe barrel 182.

The function of the ribs 122 could likewise be provided by a step in the inner surface of the body 102.

### REFERENCE NUMBERS

1 injection device
2 distal end
3 proximal end
5 housing
10 stopper
11 piston rod
12 stopper
17 abutment portion
20 body
21 sidewall
22 stepped portion
23 insert portion
24 abutment
25 proximal end
26 abutment
27 end face
28 distal end
30 protective cap
31 sidewall
32 abutment portion
33 receptacle
34 abutment
35 proximal end
36 abutment
38 distal end
39 end face
40 fastening mechanism
41 dead end stop
42 slot
43 dead end portion
44 escape portion
45 intermediate portion
46 pin
47 slot
48 pin
49 free end portion
50 activation lock
51 abutment
52 abutment
53 ring
54 segment
55 segment
56 perforated structure
57 perforated structure
58 perforated structure
59 connector
60 sleeve
61 sidewall
64 abutment
65 proximal end
68 distal end
72 flap
79 medicament container
80 syringe
81 flange
82 barrel
83 cavity
84 cavity
85 bypass
86 shoulder portion
87 abutment portion
88 outlet
90 needle
92 needle hub
94 needle shield
101 injection device
103 protective cap
104 window
110 piston rod
111 end face
112 latch
113 toothing
114 retarder
120 body
121 stop
122 rib
126 snap feature
130 spring
140 needle shield
150 clamp
151 hook
152 hook
160 sleeve
161 collar
162 window
163 spring seat
164 proximal stop
165 distal stop
166 sleeve leg
170 container carrier
171 proximal portion
172 leg
173 protrusion
174 aperture
175 leg
180 syringe
181 flange
182 barrel
183 cavity
190 needle

1-16. (canceled)

17. An injection device for expelling a dose of a medicament, the injection device comprising:
an elongated housing with a distal end and a proximal end opposite to the distal end, the elongated housing comprising a body and a protective cap,
a medicament container comprising a barrel having a first cavity sealed in a proximal direction by a first stopper slidably arranged inside the barrel, the first stopper including a proximally facing abutment portion and the barrel including a distally facing abutment portion,
wherein the protective cap is configured to abut the distally facing abutment portion of the barrel,
wherein the body is configured to abut the proximally facing abutment portion of the first stopper,
wherein the first stopper is movable in distal direction relative to the barrel from an initial position into a priming position by moving the protective cap along the proximal direction relative to the body from a preset position into an activation position.

18. The injection device according to claim 17, further comprising an activation lock detachably or movably arranged on the elongated housing and operable to block a movement of the protective cap from the preset position towards the activation position.

19. The injection device according to claim 18, wherein the activation lock is frangibly connected to at least one of the body and the protective cap.

20. The injection device according to claim 18, wherein the activation lock is connected to at least one of the body and the protective cap by at least one of a perforated structure and a structurally weakened connector comprising a predetermined breaking structure.

21. The injection device according to claim 18, wherein the activation lock comprises a ring enclosing an outside surface of the housing.

22. The injection device according to claim 18, wherein the activation lock comprises a distally facing abutment portion to abut a proximally facing abutment portion of the protective cap.

23. The injection device according to claim 18, wherein the activation lock comprises a proximally facing abutment portion to abut a distally facing abutment portion of the body.

24. The injection device according to claim 17, wherein a proximally facing abutment portion of the protective cap is configured to abut a distally facing abutment portion of the body when the protective cap is in or approaches the activation position relative to the body.

25. The injection device according to claim 17, wherein the protective cap and the body are telescopically displaceable in a longitudinal direction, wherein one of the protective cap and the body comprises a receptacle and wherein the other one of the protective cap and the body comprises an insert portion and wherein the receptacle is sized to receive the insert portion.

26. The injection device according to claim 25, wherein an activation lock is arranged on the insert portion, the activation lock being detachably or movably arranged on the elongated housing and operable to block a movement of the protective cap from the preset position towards the activation position.

27. The injection device according to claim 17, wherein the protective cap is detachably coupled to the body via a fastening mechanism, wherein the fastening mechanism is transferable from a locked configuration, in which the protective cap is in the preset position, to a release configuration, in which the protective cap is in or approaches the activation position.

28. The injection device according to claim 27, wherein the fastening mechanism comprises a slot in a sidewall of one of the protective cap and the body and wherein the fastening mechanism (40) comprises a pin protruding from the sidewall of the other one of the protective cap and the body, wherein the pin is slidably displaceable along the slot.

29. The injection device according to claim 28, wherein the slot comprises a dead end portion forming a stop for the pin and wherein the slot comprises an escape portion to enable removal of the pin from the slot and wherein the dead end portion extends at a predefined direction relative to the escape portion.

30. The injection device according to claim 17, wherein the medicament container comprises a second stopper to separate a second cavity from the first cavity.

31. The injection device according to claim 17, wherein at least one of a lyophilized medicament, a liquid medicament, a diluent and a solvent is arranged inside the medicament container.

32. The injection device according to claim 17, wherein the protective cap is detachably connected to the body.
